(19) United States
(12) Patent Application Publication
(10) Pub. No.: US 2017/0209119 A1
(43) Pub. Date: Jul. 27, 2017

(54) PHOTOACOUSTIC ULTRASONIC IMAGING APPARATUS

(71) Applicant: CANON KABUSHIKI KAISHA, Tokyo (JP)

(72) Inventors: Fumitaro MASAKI, Tokorozawa-shi (JP); Nobuhito SUEHIRA, Tokyo (JP); Kazuhiko FUKUTANI, Yokohama-shi (JP); Takuro MIYASATO, Tokyo (JP); Robert A KRUGER, Oriental, NC (US)

(21) Appl. No.: 15/411,788

(22) Filed: Jan. 20, 2017

Related U.S. Application Data
(60) Provisional application No. 62/287,819, filed on Jan. 27, 2016.

(51) Int. Cl. A61B 8/00; A61B 5/00; A61B 8/14

(52) U.S. Cl. CPC A61B 8/4416; A61B 8/14; A61B 8/44; A61B 5/0095; A61B 8/4281

(57) ABSTRACT

A photoacoustic ultrasonic imaging apparatus includes a light emitting portion configured to emit pulsed light; a photoacoustic sound source configured to absorb the pulsed light and generate photoacoustic waves to be transmitted toward a subject; a plurality of transducers configured to receive photoacoustic waves reflected by the subject and output electric signals; and an information processor configured to output a photoacoustic ultrasonic echo image of the subject by using the electric signals. The information processor is configured to calculate the photoacoustic ultrasonic echo image on the basis of positional information on the photoacoustic sound source.

### BACKGROUND OF THE INVENTION

Field of the Invention

The present disclosure relates to a photoacoustic ultrasonic imaging apparatus that images subject information by using ultrasonic waves.

Description of the Related Art

Photoacoustic imaging (PAI) is available as a technique for imaging optical absorbers (for example, blood vessels in a living body) in a subject. PAI is a technique for generating image information representing a distribution of optical absorbers by using a theory that a photoacoustic effect produced by irradiating a subject with light causes photoacoustic waves to be generated by optical absorbers. For example, PAI in which hemoglobin is used as an optical absorber enables imaging of blood vessels in a subject, as described in Zhang et al. Applied Physics Letters 90, 053901, pp. 1-3, 2007.

On the other hand, ultrasonic imaging is available as a method for representing information on the structure of a subject. In ultrasonic imaging, ultrasonic waves are transmitted from an acoustic-wave probe provided with an array of a plurality of transducers to a subject. The ultrasonic waves that have been transmitted into the subject cause reflected waves to be generated at an interface between acoustic impedances. The reflected waves are received by the acoustic-wave probe, and thereby image information regarding the acoustic impedance of the subject is generated.

In ultrasonic imaging, there is photoacoustic ultrasonic imaging in which photoacoustic waves generated by a photoacoustic effect, instead of acoustic waves generated by transducers, are used as ultrasonic waves to be transmitted to a subject. Thomas Felix Fehm, Xose Luis Dean-Ben and Daniel Razansky, Proc. of SPIE Vol. 9323 describes a hand-held probe that transmits photoacoustic waves to a subject, the photoacoustic waves being generated by irradiating a point-shaped optical absorber disposed in the probe with pulsed light, and that images a living body by using, as transmission waves, photoacoustic waves generated by using the point-shaped optical absorber as a sound source.

In photoacoustic ultrasonic imaging, a photoacoustic sound source that transmits ultrasonic waves is disposed so as to be acoustically in contact with an acoustic matching liquid that is disposed between a subject and an exciting light source. In the case of imaging a subject in such photoacoustic ultrasonic imaging, the position accuracy of image information may decrease, which may cause a problem in image fusion with an imaging result of ultrasonic imaging, photoacoustic imaging, or another modality.

### SUMMARY OF THE INVENTION

An embodiment of the present disclosure is directed to obtaining image information with high position accuracy in an apparatus that obtains information on a subject by using acoustic waves generated by a photoacoustic effect.

Note that, in this specification, positional information on a sound source is information about the position of a sound source relative to an observation system, and a subject or a subject mount unit is adopted as a reference of the observation system.

A photoacoustic ultrasonic imaging apparatus according to an embodiment includes a light emitting portion configured to emit pulsed light; a photoacoustic sound source configured to absorb the pulsed light and generate photoacoustic waves to be transmitted toward a subject; a plurality of transducers configured to receive photoacoustic waves reflected by the subject and output electric signals; and an information processor configured to output a photoacoustic ultrasonic echo image of the subject by using the electric signals. The information processor is configured to calculate the photoacoustic ultrasonic echo image on the basis of positional information on the photoacoustic sound source.

Further features of the present disclosure will become apparent from the following description of exemplary embodiments with reference to the attached drawings.

### BRIEF DESCRIPTION OF THE DRAWINGS

FIG. 1 is a diagram illustrating the configuration of a photoacoustic ultrasonic imaging apparatus according to a first embodiment.

FIG. 2 is a schematic diagram for describing generation of photoacoustic ultrasonic waves according to the first embodiment.

FIG. 3 is a diagram illustrating the configuration of a probe and the arrangement of transducers according to the first embodiment.

FIG. 4 is a characteristic diagram illustrating the frequency dependency of the reception sensitivity of the transducers.

FIGS. 5A and 5B are diagrams illustrating a photoacoustic image of a sheet-shaped photoacoustic sound source according to the first embodiment.

FIGS. 6A and 6B are diagrams illustrating a result of ultrasonic imaging of a phantom including a resin sphere portion obtained by the photoacoustic ultrasonic imaging apparatus according to the first embodiment.

FIGS. 7A and 7B are diagrams illustrating a result of ultrasonic imaging of a phantom including metal wires obtained by the photoacoustic ultrasonic imaging apparatus according to the first embodiment.

FIG. 8 is a diagram illustrating propagation of ultrasonic waves generated by the sheet-shaped photoacoustic sound source according to the first embodiment.

FIG. 9 is a diagram illustrating a sheet-shaped photoacoustic sound source including a bent portion according to a second embodiment.

### DESCRIPTION OF THE EMBODIMENTS

Hereinafter, embodiments of the present disclosure will be described with reference to the drawings. Note that the dimensions, materials, shapes, and relative positions of the components described below are to be appropriately changed in accordance with the configuration of an apparatus or various conditions to which an embodiment is applied. Thus, the scope of the invention is not limited to the description given below.

An embodiment relates to an imaging technique for transmitting ultrasonic waves to a subject, receiving ultrasonic echoes including characteristic information on the inside of the subject, and generating an ultrasonic echo image. Thus, an embodiment may be considered as an ultrasonic imaging apparatus or a control method therefor. Also, an embodiment may be considered as a program that causes an information processing apparatus including hardware resources, such as a CPU and a memory, to execute the method, or a storage medium storing the program.

Acoustic waves used in an embodiment are typically ultrasonic waves at frequencies in the range from 20 KHz to 1 GHz. Electric signals obtained through conversion of acoustic waves by transducers or the like are also referred to as acoustic signals. Note that the term “ultrasonic waves” or “acoustic waves” in the following description does not limit their wavelengths. Acoustic waves generated by a photoacoustic effect are referred to as photoacoustic waves or photoultrasonic waves.

In the following description, ultrasonic waves generated by irradiating a subject with pulsed light are referred to as photoacoustic waves unless otherwise specified. Reflected waves from a subject generated by causing ultrasonic waves to propagate through the subject are referred to as ultrasonic echoes, and reflected ultrasonic waves from a subject generated by irradiating a photoacoustic sound source with pulsed light and causing photoacoustic waves to propagate through the subject are referred to as photoacoustic ultrasonic echoes.

Furthermore, techniques for obtaining image information on a subject by using photoacoustic waves, ultrasonic echoes, and photoacoustic echoes are respectively referred to as “photoacoustic imaging”, “ultrasonic imaging”, and “photoacoustic ultrasonic imaging”.

Image information obtained by using photoacoustic waves, ultrasonic echoes, or photoacoustic echoes is referred to as an “ultrasonic echo image”. Image information on a subject obtained by using “photoacoustic waves” from a subject is referred to as a “photoacoustic image”, “ultrasonic echo image”, or “photoacoustic ultrasonic echo image”.

A photoacoustic ultrasonic imaging apparatus irradiates a photoacoustic sound source with pulsed light so as to cause photoacoustic waves generated by the photoacoustic sound source to propagate through (to be transmitted through) a subject, and obtains acoustic impedance information (characteristic information) on the subject as image information.

### First Embodiment

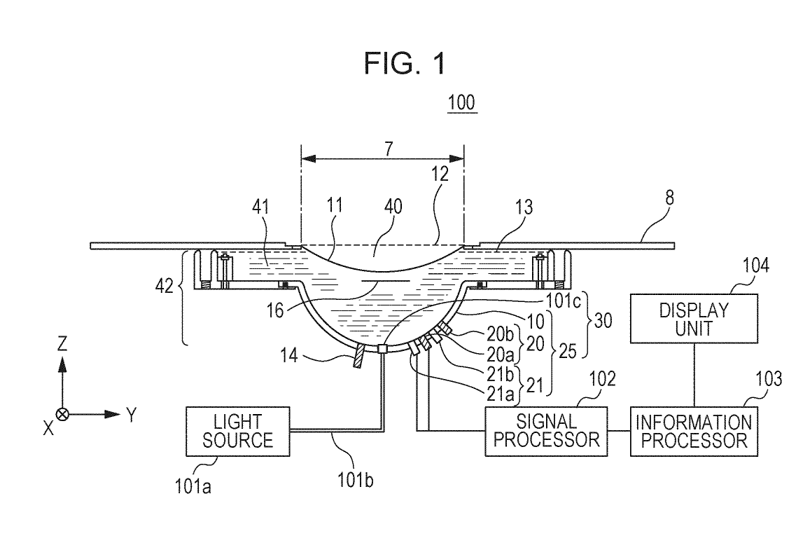

A first embodiment will be described with reference to FIGS. 1 to 8.

### Configuration of Apparatus

FIG. 1 is a diagram illustrating the schematic configuration of a photoacoustic ultrasonic imaging apparatus 100 according to the first embodiment. The craniocaudal direction of a subject is the Y direction.

A unit formed of a supporting member 10 and a plurality of transducers 20 (20a, 20b . . . ) and transducers 21 (21a, 21b . . . ) disposed on the supporting member 10 is referred to as a transducer array 25. A unit formed of the transducer array 25 and a light emitting portion 101c provided to the transducer array 25 is referred to as a probe 30. In the probe 30 of an array-type, the plurality of transducers 20 (20a, 20b . . . ) and a plurality of transducers 21 (21a, 21b . . . ) are arranged so as to be spaced from each other on a curved surface with a negative curvature of the supporting member 10, which is bowl-shaped. For the purpose of understanding, some of the transducers 20 and 21 are illustrated as representatives. The transducers 20 and 21 will be described in detail below with reference to FIG. 3.

A holding cup 11 is fixed to an examining table 8 so as to hold a subject 40 that is inserted through an opening 7 provided in the examining table 8. The upper surface of the holding cup 11 is soaked in an acoustic matching liquid 41 for the purpose of acoustic impedance matching in the gap between the subject 40 and the holding cup 11. A liquid surface 12 is the liquid surface of the acoustic matching liquid 41 on the upper surface side of the holding cup 11 and corresponds to the draft line of the subject 40. Use of the holding cup 11 enables the shape of the subject 40 to be maintained and the measurement environment to be stabilized. The liquid surface of the acoustic matching liquid 41 on the lower side of the holding cup 11 is denoted by reference numeral 13.

The acoustic matching liquid 41 is stored in a vessel 42 such that the acoustic matching liquid 41 is acoustically in contact with the transducers 20 or 21 and the subject 40 between the transducers 20 or 21 and the subject 40. As illustrated in FIG. 1, a photoacoustic sound source 16 is disposed between the transducers 20 or 21 and the subject 40 while being soaked in the acoustic matching liquid 41 stored in the vessel 42. The photoacoustic sound source 16 is a sheet-shaped photoacoustic sound source using a polyethylene sheet material and is disposed in the vessel 42 so as to be interposed between the holding cup 11 and the probe 30 by a stretching member that is not illustrated. In another embodiment, a plurality of photoacoustic sound sources 16 may be prepared for the purpose of replacing one of them with another in accordance with a subject or an imaging condition, and a mounting unit that allows an entire supporting member (not illustrated) including the photoacoustic sound source 16 to be replaced may be provided in the supporting member.

The acoustic matching liquid 41 may be, for example, water, physiological saline solution, gel, or castor oil. The subject 40 may be, for example, breasts or extremities of a living body. In a case where the subject 40 is a portion of a human body, water or aqueous solution is used as the acoustic matching liquid 41.

The state of contact between the subject 40 and each of the holding cup 11 and the acoustic matching liquid 41 is observed by a camera 14. An optical image captured by the camera 14 can be used for various kinds of control and information processing.

A light source 101a, an optical system 101b, and the light emitting portion 101c are arranged so that light emitted by the light source 101a travels through the optical system 101b and that pulsed light 15 is emitted from the light emitting portion 101c toward the photoacoustic sound source 16. The light emitting portion 101c is optically coupling with the light source 101a via the optical system including an optical fiber.

A pulse laser apparatus (for example, a Ti:S laser at a wavelength of around 800 nm) is suitable for the light source 101a from the viewpoint of output and so forth, but a flash lamp or a light-emitting diode may be used instead of a pulsed light source. The wavelength of the pulsed light 15 has an emission spectrum in the range from the near-infrared region to the infrared region (the wavelengths of 800 nm to 10 μm) and is determined in accordance with the absorbance spectrum of the photoacoustic sound source 16. Specifically, the wavelength of the pulsed light 15 is selected so that the absorbance of the photoacoustic sound source 16 is 1 or more. In other words, the optical characteristic and the optical distance (thickness) of the photoacoustic sound source 16 are selected so that the absorbance for the pulsed light 15 is 1 or more. Use of a wavelength tunable laser or the like capable of emitting light having a plurality of wavelengths as the light source 101a enables oxygen saturation to be measured.

The optical system 101b is provided to apply light with a desired shape and intensity. As the optical system 101b, for example, an optical fiber, a lens, a mirror, a prism, and a diffusion plate may be used. In FIG. 1, the light emitting portion 101c is provided at an intersection point of the rotation center axis of the bowl-shaped supporting member 10 and the supporting member 10, but the light emitting portion 101c may be provided at another position as long as the photoacoustic sound source 16 can be irradiated with light.

In this embodiment, the supporting member 10 is hemispherical and bowl-shaped. Alternatively, the supporting member 10 may have a quadric surface of revolution that has an ellipse, parabola, hyperbola, or the like at a part of the cross section. If the supporting member 10 has a quadric surface of revolution and if the transducers 20 and the light emitting portion 101c are disposed on the inner surface having a negative curvature, the symmetry among a light irradiation region, an acoustic wave propagation region, and a subject is ensured, and a wide field of view (FOV) is ensured.

Each of the transducers 20 or 21 outputs an analog electric signal in response to receipt of an ultrasonic wave. A signal processor 102 performs amplification, digital conversion, or correction on the analog electric signal when necessary and outputs a resulting signal. The digital electric signal that has been output is input to an information processor 103 directly or through a memory (not illustrated). The information processor 103 is an information processing device including arithmetic resources such as a CPU and a storage device. For example, a processing circuit, a work station, or a personal computer (PC) is used as the information processor 103. The information processor 103 reconstructs the digital electric signal to generate image information and outputs the image information to a display unit 104. A method according to the related art such as phasing addition, Fourier transform, or back projection may be used for image reconstruction. The display unit 104 may be separated from the photoacoustic ultrasonic imaging apparatus 100. A display device such as a liquid crystal display or plasma display may be used as the display unit 104.

An image reconstruction method for photoacoustic imaging is described in, for example, Wang et al. Phys. Med. Biol. 2004; 49; 3117-3124.

$$A ( r ) dS = \frac{4 π {C}_{p} kt}{β} IFFT ( \frac{{P}_{i} ( ω )}{{P}_{0} ( ω )} ) \qquad Equation 1$$

Here, A(r) represents a distribution of optical absorption, and the integral on the left side represents projection of the distribution of optical absorption A(r) onto a spherical surface at a distance ct from an ultrasonic transducer. r represents a position in a space in a subject for which the distribution of optical absorption is obtained, and r₁ represents the position of the ultrasonic transducer. Cₚ, β, c, and k represent proportionality coefficients that depend on heat capacity, thermal expansion coefficient, sonic speed, and illumination condition, respectively. p₀(t) represents time dependency of a photoacoustic signal from a point sound source, and p₁(t) represents time dependency of a photoacoustic signal from a subject. P₀(ω) and P₁(ω) represent Fourier transform of p₀(t) and p₁(t), respectively.

As expressed by Equation 1, the position r₁ of the transducer 20 has an influence on an imaging result in photoacoustic imaging.

### Generation of Photoacoustic Ultrasonic Waves

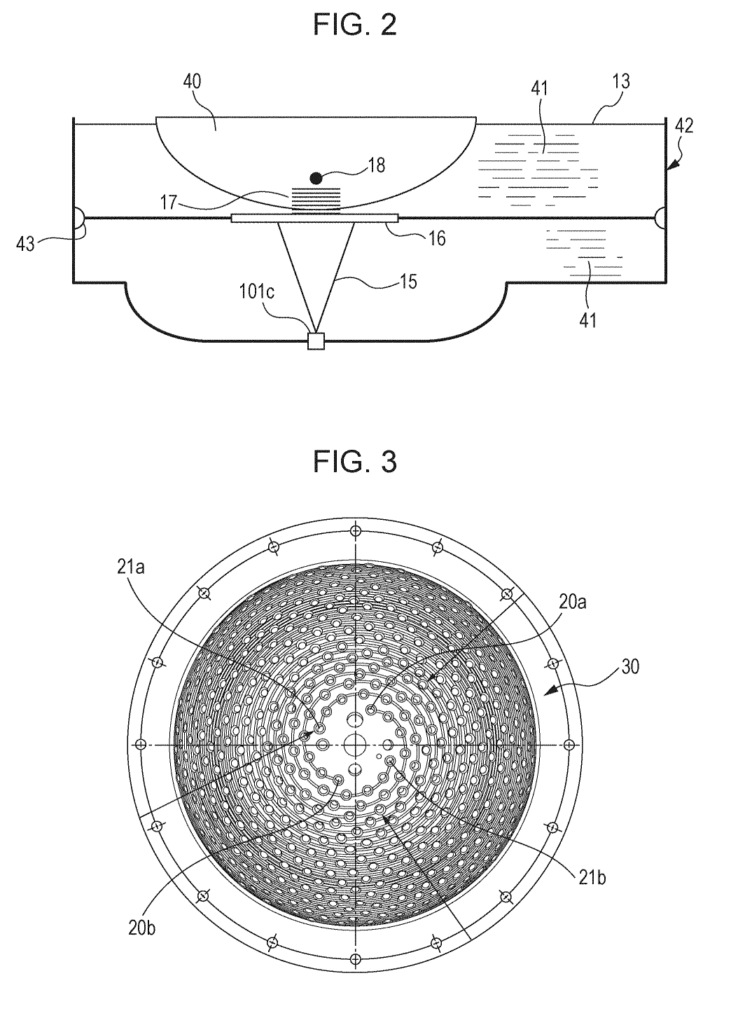

FIG. 2 is a schematic diagram for describing generation of photoacoustic ultrasonic waves. Planar photoacoustic ultrasonic waves 17 are generated by a photoacoustic effect from the sheet-shaped photoacoustic sound source 16 that has absorbed the pulsed light 15 emitted from the light emitting portion 101c.

The photoacoustic sound source 16 according to this embodiment is an optical absorption sheet including a portion that contains an optical absorber composed of carbon black and extends in a sheet-shape. A high-density polyethylene sheet containing black paint may be used as the material of the photoacoustic sound source 16. When such a sheet is formed with a thickness of about 0.1 mm and is disposed on an optical path of the pulsed light 15, the sheet absorbs 99.9% or more of the pulsed light 15.

The photoacoustic sound source 16 extends in a direction that crosses the irradiation direction of the pulsed light 15 emitted from the light emitting portion 101c. The photoacoustic sound source 16 is disposed such that the subject 40 is not directly viewed from the light emitting portion 101c, and thus interference of ultrasonic waves (not illustrated) generated by the subject 40 with the photoacoustic ultrasonic waves 17 generated by the photoacoustic sound source 16 can be suppressed.

The photoacoustic sound source 16 is not necessarily a sheet-shaped sound source, but may have a point-shape, a line-shape, a plane-shape, or a combination of the shapes when the subject 40 is viewed from the light emitting portion 101c.

The sheet-shaped photoacoustic sound source 16 is stretched by a stretching member 43 in the vessel 42 so as to separate the acoustic matching liquid 41 into a portion that is acoustically in contact with the transducers 20 or 21 and a portion that is acoustically in contact with the subject 40, as illustrated in FIG. 2.

The photoacoustic sound source 16 may have a function of absorbing the pulsed light 15 emitted from the light emitting portion 101c so as to expand and contract and generating pressure waves. That is, the photoacoustic sound source 16 may have an absorbance of 0.1 or more (a spectral transmittance of 79% or less) defined by the Lambert-Beer law with respect to the pulsed light 15 emitted from the light emitting portion 101c. In another embodiment, the photoacoustic sound source 16 may have an absorbance of 0.5 or more (a spectral transmittance of 31.6% or less). In still another embodiment, the photoacoustic sound source 16 may have an absorbance of 1 or more (a spectral transmittance of 10% or less). This means that the thickness of the sheet-shaped photoacoustic sound source 16 has a lower limit for the purpose of ensuring an effective thickness of a photoacoustic conversion layer and ensuring photoacoustic output.

In the photoacoustic sound source 16, the photoacoustic conversion layer corresponds to a penetration depth λp of the pulsed light 15. Thus, if the photoacoustic sound source 16 is too thick relative to the penetration depth λp of the pulsed light 15, an inertial weight that does not contribute to vibration is produced and the photoacoustic conversion efficiency decreases. That is, the photoacoustic sound source 16 may have an absorbance of 6 or less (a spectral transmittance of 0.0001% or less) defined by the Lambert-Beer law with respect to the pulsed light 15 emitted from the light emitting portion 101c. In another embodiment, the photoacoustic sound source 16 may have an absorbance of 3 or less (a spectral transmittance of 0.1% or less). This means that the thickness of the sheet-shaped photoacoustic sound source 16 has an upper limit for the purpose of reducing the weight of a vibration body other than the effective photoacoustic conversion layer and ensuring photoacoustic output.

Thus, when the thickness of the photoacoustic sound source 16 in the direction along the propagation direction of the pulsed light 15 is represented by t and when the penetration depth of the pulsed light 15 emitted from the light emitting portion 101c in the direction along the propagation direction is represented by λp, the photoacoustic sound source 16 may satisfy the following general formula 1.

0.1≦λp/t≦6 General formula 1

In another embodiment, the photoacoustic sound source 16 may satisfy the following general formula 2.

0.5≦λp/t≦3 General formula 2

The material applied to the photoacoustic sound source 16 is not limited to a polyethylene sheet, and various materials may be used, for example, acrylic, polyester, or polyvinyl chloride. In the case of the positional relationship illustrated in FIG. 1, where the photoacoustic sound source 16 is disposed between the probe 30 and the subject 40, the photoacoustic sound source 16 may be made of a material having an acoustic impedance characteristic that allows ultrasonic waves reflected and scattered in the subject 40 to pass therethrough. If the photoacoustic sound source 16 absorbs the ultrasonic waves reflected and scattered in the subject 40, the acoustic-wave energy that reaches the probe 30 decreases.

The optical absorber contained in the photoacoustic sound source 16 may be graphite (carbon black) or another pigment. The thickness of the photoacoustic sound source 16 is determined in accordance with the center frequency of the photoacoustic ultrasonic waves 17 and the frequency characteristic of the reception sensitivity of the transducers 20. If the photoacoustic sound source 16 is sheet-shaped as in this embodiment, an increase in the sheet thickness causes shift to a lower frequency and a decrease in the sheet thickness causes shift to a higher frequency. In this way, the reception characteristic of photoacoustic imaging is changed by adjusting the shape (thickness) of the photoacoustic sound source 16, and thus a plurality of photoacoustic sound sources 16 having different acoustic and optical characteristics may be prepared for replacement according to the subject 40 or an observation condition.

As in ultrasonic imaging according to the related art, photoacoustic ultrasonic waves propagate in a living body and are reflected and scattered at an interface where a difference exists in acoustic impedance in the living body. An example of a reflector and a scatterer includes a minute lime 18, which is a prodrome of breast cancer, as illustrated in FIG. 2.

In this embodiment, as described above, reflected and scattered ultrasonic waves derived from the planar photoacoustic ultrasonic waves 17 generated by the photoacoustic sound source 16 are received by the probe 30 that is provided with a transducer array 25. By using the probe 30, an area image of an effective reception region corresponding to the reception direction ranges of individual transducers can be collectively obtained through transmission of photoacoustic ultrasonic waves generated by single excitation of pulsed light.

### Probe and Transducers

Hereinafter, the configuration of the probe 30 will be described. In an apparatus that generates a three-dimensional image of a subject by reconstructing reflected and scattered ultrasonic waves in a subject, transducers may be disposed on a quadric surface of revolution that surrounds the subject in order to accurately obtain a distribution of sound pressure.

In particular, if a plurality of transducers are disposed on a spherical surface, a region where acoustic waves can be evenly detected from any directions can be formed in a subject, and accordingly the homogeneity of the contrast of a reconstructed image increases. The arrangement of transducers in the probe 30 is appropriately selected with reference to the scanning method for the probe 30.

FIG. 3 is a top view of a detection surface of the probe 30 viewed from the holding cup 11 side. A plurality of transducers 20 (20a, 20b . . . ) and 21 (21a, 21b . . . ) are provided on the detection surface. The plurality of transducers 20 are arranged in a spiral manner and have a center frequency of 2 MHz. The plurality of transducers 21 are arranged in a spiral manner and have a center frequency of 5 MHz. Piezoelectric elements, capacitive micromachined ultrasonic transducers (cMUT), Fabry-Perot sensors, or the like may be used as the transducers.

The transducers 20 or 21 according to this embodiment are configured to receive ultrasonic waves in a frequency band including the center frequency ±about 50%. Thus, in this embodiment, two types of transducers 20 and 21 having different center frequencies are arranged on the detection surface in a rotationally symmetric manner. Accordingly, ultrasonic waves in a wide frequency band can be received and the process of three-dimensional reconstruction is simplified.

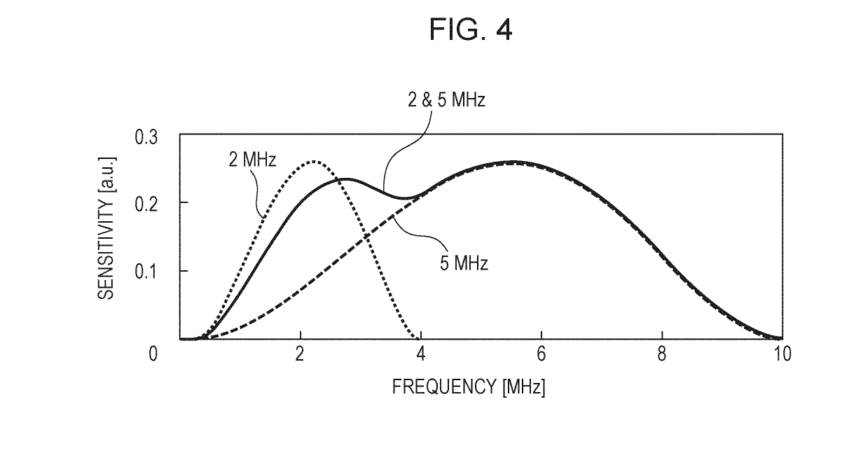

FIG. 4 illustrates a frequency band that can be covered by a combination of two types of transducers 20 and 21 having different center frequencies. The sensitivity of the transducer 20 is represented by a dotted line, whereas the sensitivity of the transducer 21 is represented by a broken line. The solid line represents the frequency band that can be covered by the combination thereof. Furthermore, even if the sensitivity of a transducer varies according to a frequency region, a relatively even sensitivity can be obtained in a wide frequency band by adjusting the output gain at the conversion of an electric signal. For example, in FIG. 4, the transducer on the high-frequency side has a low sensitivity, and thus the output gain at the conversion of an electric signal of the transducer on the high-frequency side may be increased (for example, doubled). The sensitivities of the transducers can be made constant if a gain controller (not illustrated) adjusts the output gain. An existing gain controller circuit may be used as the gain controller. Alternatively, three or more types of transducers may be used.

For reconstruction of photoacoustic ultrasonic imaging, a reconstruction algorithm common to the above-described photoacoustic imaging can be used. However, it is necessary to consider the time taken for photoacoustic ultrasonic waves to propagate from a photoacoustic sound source to a reflector or scatterer and the time taken for the photoacoustic ultrasonic waves to propagate from the reflector or scatterer to transducers, and thus the following equation is used.

$$R ( r ) dE = {Kt}^{*} IFFT ( \frac{{S}_{i} ( ω )}{{S}_{0} ( ω )} ) \qquad Equation 2$$

R(r) represents a distribution of reflection and scattering of ultrasonic waves, and the integral on the left side represents projection of the distribution R(r) onto an elliptic surface with r₁ and r₀ being a focus. Here, r represents a point in a space of a subject for which the distribution of optical absorption is obtained, r₁ represents the position of an ultrasonic transducer, and r₀ represents the position of a sound source that transmits ultrasonic waves. Cₚ, β, c, and k represent proportionality coefficients that depend on heat capacity, thermal expansion coefficient, sonic speed, and illumination condition, respectively. s₀(t) represents time dependency of a reflected and scattered ultrasonic signal in a case where a point sound source is a target, whereas s₁(t) represents time dependency of a reflected and scattered ultrasonic signal from a subject. S₀(ω) and S₁(ω) represent Fourier transform of s₀(t) and s₁(t), respectively.

As expressed by Equation 2, in ultrasonic imaging, the position of an ultrasonic transducer and the position r₀ of the sound source have an influence on the performance of the imaging. For the position r₁ of the ultrasonic transducer, position information identified before or after photoacoustic ultrasonic imaging can be used, that is, can be handled as a known value. On the other hand, the photoacoustic sound source 16 has a small weight as a sound source and is soaked in an acoustic matching liquid and thus may be moved during an observation period of photoacoustic ultrasonic imaging. Thus, a calibration method for identifying the position r₀ of the sound source is demanded. An embodiment is directed to identifying the position of the photoacoustic sound source disposed in an acoustic matching liquid and ensuring the position accuracy of a photoacoustic ultrasonic image.

First, the positions of the transducers 20 or 21 in the probe 30 are measured to perform calibration in a state where the member that generates photoacoustic ultrasonic waves (photoacoustic sound source 16) is removed from the optical path of the pulsed light 15.

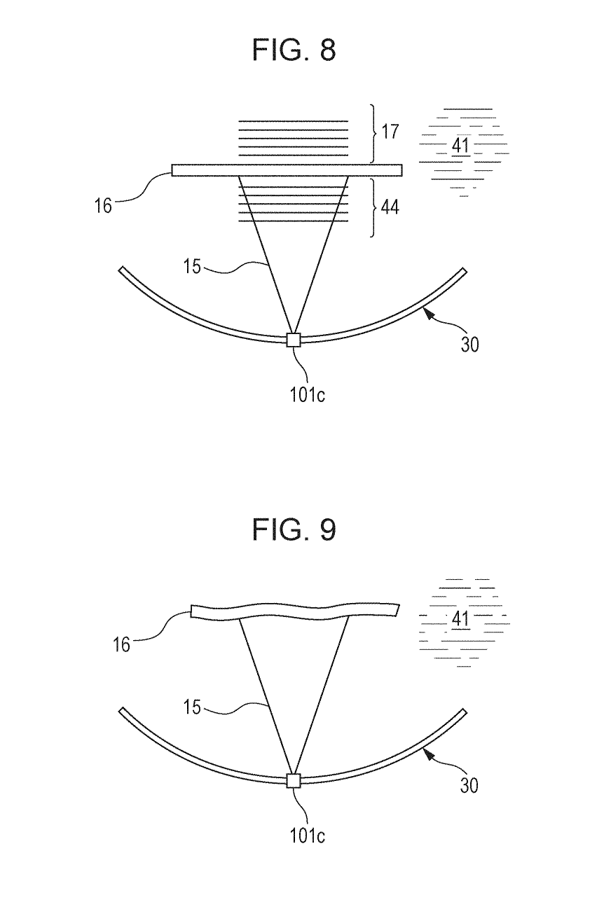

Subsequently, the photoacoustic sound source 16 is disposed on the optical path of the pulsed light 15. The photoacoustic sound source 16 according to this embodiment is formed of a polyethylene sheet. The photoacoustic ultrasonic imaging apparatus captures an image of the photoacoustic sound source 16. The photoacoustic waves from the sheet-shaped photoacoustic sound source 16 include the photoacoustic waves 17 travelling toward the subject and photoacoustic waves 44 travelling toward the probe 30, as illustrated in FIG. 8. The photoacoustic waves 44 are directly detected by the probe 30 without interacting with the subject 40. With use of the photoacoustic waves 44, the position of the photoacoustic sound source 16 is specified based on a photoacoustic image that is obtained by operating the photoacoustic ultrasonic imaging apparatus 100 in a photoacoustic imaging mode.

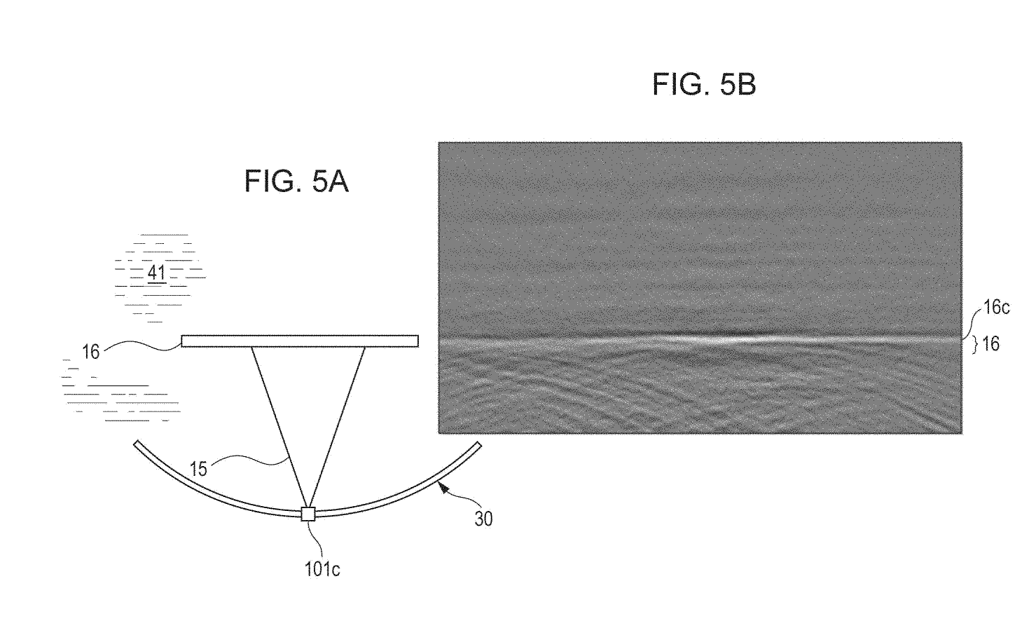

FIGS. 5A and 5B illustrate a result of capturing a photoacoustic image of the photoacoustic sound source 16 by the photoacoustic ultrasonic imaging apparatus 100 according to this embodiment. The photoacoustic sound source 16 immersed in the acoustic matching liquid 41 and the optical emitting portion 101c facing the photoacoustic sound source 16 and provided in the probe 30 are described in FIG. 5A. The bowl shaped probe 30 is immersed in the acoustic matching liquid 41. With this image capturing result, a sound source center 16c of the photoacoustic sound source 16 is calibrated. In other words, the photoacoustic image illustrated in FIG. 5B includes shape information on the photoacoustic sound source 16 including the sound source center 16c of the photoacoustic sound source 16.

In general, the sound source center 16c of the photoacoustic sound source 16 does not necessarily match the physical center in the sheet thickness direction of the photoacoustic sound source 16. It is assumed that the photoacoustic sound source 16 has a sufficiently large sheet thickness (λp/t<<1) and that the entire pulsed light 15 is absorbed near the front surface on the light emitting portion 101c side. In this case, ultrasonic waves are generated from a place where the light has been absorbed. In reconstruction of ultrasonic imaging, a position where sound waves are generated, not the physical center of the sheet thickness of the photoacoustic sound source 16, is used as a parameter of reconstruction. The position of the sound source center 16c of the photoacoustic sound source 16 is defined as a center-of-gravity position where photoacoustic ultrasonic waves are generated.

The photoacoustic sound source 16 needs to have a sheet thickness for absorbing substantially entire light. If the absorbance for the pulsed light 15 is 0.83, about 85% of the light is absorbed and 15% of the light is transmitted. If the sheet thickness of the photoacoustic sound source 16 is too small relative to the linear absorption coefficient of the pulsed light 15, for example, if the sheet has an absorption coefficient of the pulsed light 15, the pulsed light 15 passes through the sheet and reaches the living body. The light that has reached the living body generates undesired acoustic waves and degrades image capturing performance. A thickness of at least about one tenth of the linear absorption coefficient is necessary. In another embodiment, a thickness of at least about a half of the linear absorption coefficient is necessary.

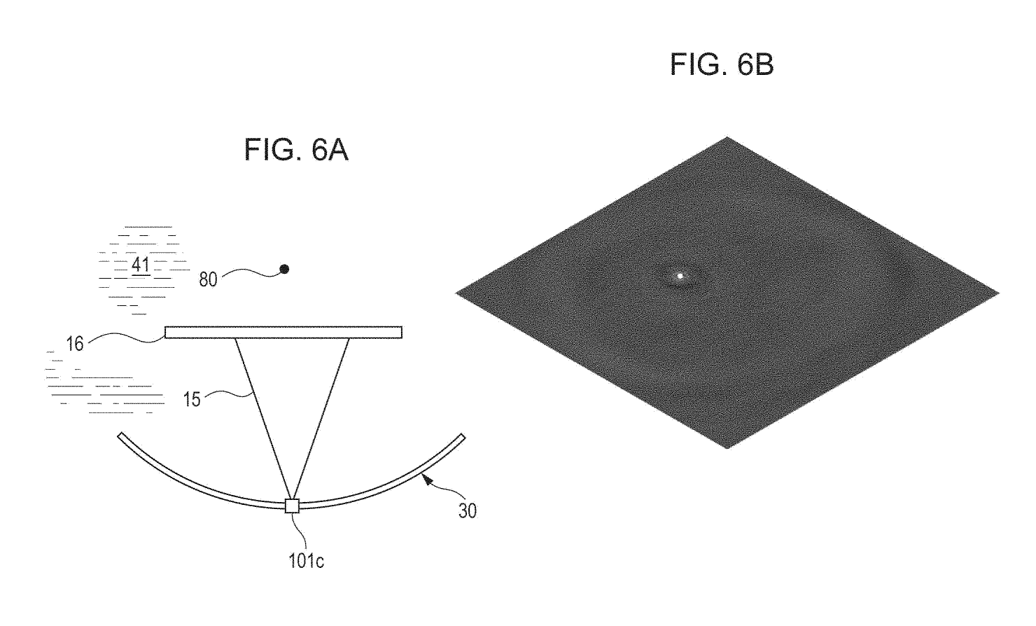

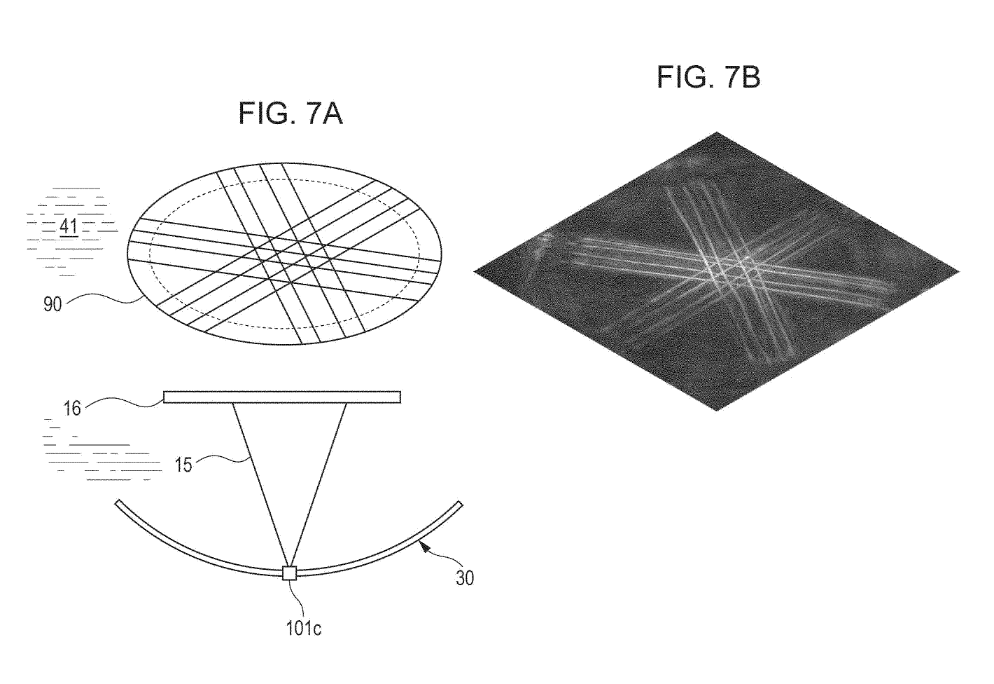

FIG. 6A illustrates a plastic sphere 80, the sheet shaped photoacoustic sound source 16 immersed in the acoustic matching liquid 41 and the optical emitting portion 101c provided in the probe 30 facing the photoacoustic sound source 16. The plastic sphere 80 and the bowl shaped probe 30 are located in the acoustic matching liquid 41. FIG. 6B illustrates a result of capturing an image of a point scatterer provided in a phantom of a living body by using the photoacoustic ultrasonic imaging apparatus 100 according to this embodiment. FIG. 7A illustrates twelve wires 90, the sheet shaped photoacoustic sound source 16 immersed in the acoustic matching liquid 41 and the optical emitting portion 101c provided in the probe 30 facing the photoacoustic sound source 16. The twelve wires 90 and the bowl shaped probe 30 are located in the acoustic matching liquid 41. FIG. 7B illustrates a result of capturing an image of a wire scatterer, instead of the point scatterer. The positions of the individual scatterers can be fixed by a certain supporting mechanism, such as a frame member, wire, or supporting film. As illustrated in FIGS. 6B and 7B, image information representing the characteristic information on a subject can be generated by using planar photoacoustic ultrasonic waves generated by a photoacoustic effect, as in this embodiment.

FIG. 8 illustrates the sheet shaped photoacoustic sound source 16 immersed in the acoustic matching liquid 41 and the optical emitting portion 101c provided in the probe 30 facing the photoacoustic sound source 16. The twelve wires 90 and the bowl shaped probe 30 are located in the acoustic matching liquid 41. The time to calibrate the sheet thickness position of the photoacoustic sound source 16 is not necessarily before image capturing of a subject. The calibration may be performed at the same time when an image of a living body is captured. As described above, photoacoustic waves generated by the sheet-shaped photoacoustic sound source 16 include photoacoustic waves directly detected, such as the photoacoustic waves 44, in addition to photoacoustic waves that travel toward a subject, are scattered by a scatterer in the subject, and are then detected by ultrasonic transducers, such as the photoacoustic waves 17 illustrated in FIG. 8. These signals detected by the ultrasonic transducers have sufficient temporal differences and thus can be separated from one another. As a result of imaging the signals of the photoacoustic waves 44 using photoacoustic imaging, the position of the sheet-shaped photoacoustic sound source 16 can be constantly calculated and used for reconstruction of ultrasonic imaging.

The photoacoustic image illustrated in FIG. 5B is based on the photoacoustic waves 44 that propagate toward the plurality of transducers 21 without propagating toward the subject, among the photoacoustic waves 17 and 44 generated by the photoacoustic sound source 16 as a result of irradiating the photoacoustic sound source 16 with the pulsed light 15 emitted from the light emitting portion 101c, as illustrated in FIG. 8.

That is, positional information on the photoacoustic sound source 16 is determined from a photoacoustic image that is obtained by receiving, with the plurality of transducers 21, the photoacoustic waves 44 that do not propagate toward the subject and that propagate toward the plurality of transducers 21 among the photoacoustic waves 17 and 44 generated by the photoacoustic sound source 16 illustrated in FIG. 8. In other words, positional information on the photoacoustic sound source 16 is determined from a photoacoustic image that is obtained by receiving, with the plurality of transducers 21, the photoacoustic waves 44 that directly propagate toward the plurality of transducers 21 among the photoacoustic waves 17 and 44 generated by the photoacoustic sound source 16 illustrated in FIG. 8.

FIG. 9 illustrates the warped photoacoustic sound source 16 immersed in the acoustic matching liquid 41 and the optical emitting portion 101c provided in the probe 30 facing the photoacoustic sound source 16. The twelve wires 90 and the bowl shaped probe 30 are located in the acoustic matching liquid 41. The sheet-shaped photoacoustic sound source 16 used as a sound source is not necessarily completely planar. The photoacoustic sound source 16 may be in a form that the sheet surface is bent as illustrated in FIG. 9, or may be in a form that the sheet surface has fine convex and concave portions (not illustrated). As a result of performing reconstruction of ultrasonic imaging by reflecting the shape of the sheet surface of the photoacoustic sound source 16, higher-performance ultrasonic imaging can be performed.

In this embodiment, the sheet-shaped photoacoustic sound source 16 is used as an absorber of pulsed light, but the shape of the absorber is not limited to a sheet-shape. The position of the sound source can be calibrated by using the method according to an embodiment even if the absorber is spherical or cylindrical. Furthermore, the photoacoustic sound source may have a configuration in which only a part of a sheet absorbs light, or may be a combination of a plurality of spheres or cylinders.

The photoacoustic ultrasonic imaging apparatus according to an embodiment may have a photoacoustic imaging mode in which a photoacoustic image of a subject is obtained in a state where a photoacoustic sound source is not disposed on an irradiation path of pulsed light. In an imaging apparatus having both an ultrasonic imaging mode and a photoacoustic imaging mode, different pieces of characteristic information can be associated with each other through image fusion. According to an embodiment, image fusion of a photoacoustic ultrasonic image and a photoacoustic image can be performed in which the position accuracy included in image information is ensured.

### Second Embodiment

In a case where the sheet-shaped photoacoustic sound source 16 is used, the photoacoustic ultrasonic waves generated thereby are planar waves. The region outside the region irradiated with the photoacoustic ultrasonic waves is not imaged. The area of this region is determined in accordance with the size of the sheet-shaped photoacoustic sound source 16. However, an imaging region can be extended by scanning the probe 30, the light emitting portion 101c, and the sheet-shaped photoacoustic sound source 16 with respect to a subject.

Thus, a configuration in which a scanning mechanism is provided to extend the imaging region, such as the measurement unit 3 described in International Patent Application Publication No. WO/2015/162896, is also included in an embodiment. As the scanning mechanism, an XY stage according to the related art is applicable. As scanning tracks, those of spiral scanning, raster scanning, and boustrophedon scanning may be used.

The sheet-shaped photoacoustic sound source 16 is disposed in an acoustic matching liquid for acoustic impedance matching. Thus, the position and shape of the sheet-shaped photoacoustic sound source 16 may be changed due to the inertia (inertial weight) of the acoustic matching liquid during a scanning operation for obtaining photoacoustic ultrasonic imaging. In the case of extending an imaging region by repeating scanning and image capturing, high-performance ultrasonic imaging can be performed by calibrating the center position of the sheet-shaped photoacoustic sound source 16 every time image capturing is performed. A mechanism for collectively moving the plurality of transducers 20 relative to the photoacoustic sound source 16 may further be provided. In this case, the position of the photoacoustic sound source 16 is identified in accordance with a plurality of positional conditions of the plurality of transducers 20 with respect to the photoacoustic sound source 16, and thereby the identification accuracy of the positional information on the photoacoustic sound source 16 is further enhanced.

### Other Embodiments

An embodiment is also implemented by executing the following process. That is, a program implementing one or more functions of the foregoing embodiments is supplied to a system or an apparatus through a network or various types of storage media, and one or more processors in a computer included in the system or the apparatus read and execute the program. Alternatively, an embodiment may be implemented by a circuit implementing the one or more functions (for example, FPGA or ASIC).

According to an embodiment, in a photoacoustic ultrasonic imaging apparatus that obtains an acoustic impedance image of a subject by using a photoacoustic sound source, image information with higher position accuracy can be obtained.

While the present disclosure has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of U.S. Provisional Patent Application No. 62/287,819, filed Jan. 27, 2016, which is hereby incorporated by reference herein in its entirety.

What is claimed is:

1. A photoacoustic ultrasonic imaging apparatus comprising:
a light emitting portion configured to emit pulsed light;
a photoacoustic sound source configured to absorb the pulsed light and generate photoacoustic waves to be transmitted toward a subject;
a plurality of transducers configured to receive photoacoustic waves reflected by the subject and output electric signals; and
an information processor configured to output a photoacoustic ultrasonic echo image of the subject by using the electric signals,
wherein the information processor is configured to calculate the photoacoustic ultrasonic echo image on the basis of positional information on the photoacoustic sound source.

2. The photoacoustic ultrasonic imaging apparatus according to claim 1, wherein the positional information on the photoacoustic sound source is determined from a photoacoustic image of the photoacoustic sound source, the photoacoustic image being obtained by irradiating the photoacoustic sound source with the pulsed light.

3. The photoacoustic ultrasonic imaging apparatus according to claim 1, wherein the photoacoustic image includes shape information on the photoacoustic sound source.

4. The photoacoustic ultrasonic imaging apparatus according to claim 1, wherein the photoacoustic sound source is an optical absorption sheet including a portion that contains an optical absorber and extends in a sheet-shape.

5. The photoacoustic ultrasonic imaging apparatus according to claim 4, wherein the optical absorption sheet includes a portion that extends so as to be distanced from both the light emitting portion and the subject.

6. The photoacoustic ultrasonic imaging apparatus according to claim 4, wherein the optical absorption sheet is disposed such that the subject is not directly viewed from the light emitting portion.

7. The photoacoustic ultrasonic imaging apparatus according to claim 1, wherein the photoacoustic sound source has a point-shape, a line-shape, a plane-shape, or a combination of the point-shape, the line-shape, and the plane-shape, and extends in a direction that crosses an irradiation direction of the pulsed light emitted from the light emitting portion, when the subject is viewed from the light emitting portion.

8. The photoacoustic ultrasonic imaging apparatus according to claim 1, further comprising:
a vessel configured to store an acoustic matching liquid such that the acoustic matching liquid is acoustically in contact with the transducers and the subject between the transducers and the subject,
wherein the photoacoustic sound source is disposed between the transducers and the subject while being soaked in the acoustic matching liquid stored in the vessel.

9. The photoacoustic ultrasonic imaging apparatus according to claim 1, further comprising:
a vessel configured to store an acoustic matching liquid such that the acoustic matching liquid is acoustically in contact with the transducers and the subject between the transducers and the subject,
wherein the photoacoustic sound source is sheet-shaped and is disposed such that a sheet surface of the photoacoustic sound source crosses an irradiation direction of the pulsed light.

10. The photoacoustic ultrasonic imaging apparatus according to claim 9, wherein the sheet surface of the photoacoustic sound source that is sheet-shaped is stretched by a stretching member fixed to at least either of the subject and the transducers.

11. The photoacoustic ultrasonic imaging apparatus according to claim 9, wherein the photoacoustic sound source that is sheet-shaped is disposed in the vessel so as to separate the acoustic matching liquid into a portion that is acoustically in contact with the transducers and a portion that is acoustically in contact with the subject.

12. The photoacoustic ultrasonic imaging apparatus according to claim 1, wherein the plurality of transducers are fixed to a common supporting member to which the light emitting portion is fixed.

13. The photoacoustic ultrasonic imaging apparatus according to claim 1, wherein a position of the photoacoustic sound source is a center-of-gravity position where the photoacoustic waves are generated in an irradiation direction of the pulsed light emitted from the light emitting portion toward the photoacoustic sound source.

14. The photoacoustic ultrasonic imaging apparatus according to claim 1, wherein the photoacoustic sound source satisfies a general formula 0.1≦λp/t≦6, where t represents a thickness of the photoacoustic sound source in a direction along a propagation direction of the pulsed light, and λp represents a penetration depth, in the direction along the propagation direction, of the pulsed light emitted from the light emitting portion.

15. The photoacoustic ultrasonic imaging apparatus according to claim 14, wherein the photoacoustic sound source satisfies a general formula 0.5≦λp/t≦3.

16. The photoacoustic ultrasonic imaging apparatus according to claim 1, further comprising:
a mechanism configured to collectively move the plurality of transducers relative to the photoacoustic sound source,
wherein a position of the photoacoustic sound source is identified in accordance with a plurality of positional conditions of the plurality of transducers with respect to the photoacoustic sound source.

17. The photoacoustic ultrasonic imaging apparatus according to claim 1, wherein the pulsed light has a spectrum in a range from a near-infrared region to an infrared region, and the photoacoustic sound source has an absorbance spectrum at an absorbance of 1 or more for the pulsed light.

18. The photoacoustic ultrasonic imaging apparatus according to claim 2, wherein the photoacoustic image is obtained by receiving, with the plurality of transducers, photoacoustic waves that do not propagate toward the subject and that propagate toward the plurality of transducers among photoacoustic waves generated by the photoacoustic sound source as a result of irradiating the photoacoustic sound source with the pulsed light emitted from the light emitting portion.

19. The photoacoustic ultrasonic imaging apparatus according to claim 2, wherein the photoacoustic image is obtained by receiving, with the plurality of transducers, photoacoustic waves that directly propagate toward the plurality of transducers among photoacoustic waves generated by the photoacoustic sound source as a result of irradiating the photoacoustic sound source with the pulsed light emitted from the light emitting portion.
